SEQUENCE LISTING (13 sequences)

SEQ ID NO: 1 - DNA, 19 bp, Homo sapiens
gcagctggctccctatgct

SEQ ID NO: 2 - DNA, 19 bp, Homo sapiens
ggaaggtcaggccctcaag

SEQ ID NO: 3 - DNA, 24 bp, Homo sapiens
cacgcaggagaagctcaaccacca

SEQ ID NO: 4 - DNA, 19 bp, Mus musculus
gggtgcacaacaagctggt

SEQ ID NO: 5 - DNA, 22 bp, Mus musculus
ccctctcagtttccttggctag

SEQ ID NO: 6 - DNA, 26 bp, Mus musculus
ccctttgtcgtacagctgagtgggca

SEQ ID NO: 7 - DNA, 20 bp, Homo sapiens
gatgagtatgcctgccgtgt

SEQ ID NO: 8 - DNA, 20 bp, Homo sapiens
ggcatcttcaacctccatga

SEQ ID NO: 9 - DNA, 21 bp, Homo sapiens
aaccatgtgactttgtcacag

SEQ ID NO: 10 - DNA, 15 bp, Mus musculus
acgccacccaccgga

SEQ ID NO: 11 - DNA, 19 bp, Mus musculus
ggcgggtggaactgtgtta

SEQ ID NO: 12 - DNA, 29 bp, Mus musculus
tgggaagccgaacatactgaactgctacg

SEQ ID NO: 13 - DNA, 1460 bp, Homo sapiens
tgcagcgcaggtgagctctcctgaggacctctctgtcagctcccctgattgtagggaggatccagtgtggcaagaaactcctccagcccagcaagcagctcaggatgttcctgaaggccgtggtcctgaccctggccctggtggctgtcgccggagccagggctgaggtcagtgctgaccaggtggccacggtgatgtgggactacttcagccagctgagcaacaatgccaaggaggccgtggaacatctccagaaatctgaactcacccagcaactcaatgccctcttccaggacaaacttggagaagtgaacacttacgcaggtgacctgcagaagaagctggtgccctttgccaccgagctgcatgaacgcctggccaaggactcggagaaactgaaggaggagattgggaaggagctggaggagctgagggcccggctgctgccccatgccaatgaggtgagccagaagatcggggacaacctgcgagagcttcagcagcgcctggagccctacgcggaccagctgcgcacccaggtcagcacgcaggccgagcagctgcggcgccagctgaccccctacgcacagcgcatggagagagtgctgcgggagaacgccgacagcctgcaggcctcgctgaggccccacgccgacgagctcaaggccaagatcgaccagaacgtggaggagctcaagggacgccttacgccctacgctgacgaattcaaagtcaagattgaccagaccgtggaggagctgcgccgcagcctggctccctatgctcaggacacgcaggagaagctcaaccaccagcttgagggcctgaccttccagatgaagaagaacgccgaggagctcaaggccaggatctcggccagtgccgaggagctgcggcagaggctggcgcccttggccgaggacgtgcgtggcaacctgaggggcaacaccgaggggctgcagaagtcactggcagagctgggtgggcacctggaccagcaggtggaggagttccgacgccgggtggagccctacggggaaaacttcaacaaagccctggtgcagcagatggaacagctcaggcagaaactgggcccccatgcgggggacgtggaaggccacttgagcttcctggagaaggacctgagggacaaggtcaactccttcttcagcaccttcaaggagaaagagagccaggacaagactctctccctccctgagctggagcaacagcaggaacagcagcaggagcagcagcaggagcaggtgcagatgctggcccctttggagagctgagctgcccctggtgcactggccccaccctcgtggacacctgccctgccctgccacctgtctgtctgtctgtcccaaagaagttctggtatgaacttgaggacacatgtccagtgggaggtgagaccacctctcaatattcaataaagctgctgagaatctagcctc

(19) United States
(12) Patent Application Publication
(10) Pub. No.: US 2009/0220958 A1
(43) Pub. Date: Sep. 3, 2009

(54) METHOD FOR DIAGNOSING HEPATIC DISEASES AND SCREENING METHOD OF MOLECULES FOR TREATMENT OF HEPATIC DISEASE

(75) Inventors: Pierre Casellas, Paris (FR); Hubert Vidal, Paris (FR); Isabelle Buisson, Paris (FR); Jean Marchand, Paris (FR); Dominique Simon, Paris (FR); Sylvaine Galiegue, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/092,123

(22) PCT Filed: Nov. 10, 2006
(86) PCT No.: PCT/FR2006/002503

§ 371 Date: Dec. 12, 2008

(30) Foreign Application Priority Data
Nov. 10, 2005 (FR) 0511432

(51) Int. Cl. C12Q 1/68; G01N 33/566; G01N 33/534

(52) U.S. Cl. 435/6; 435/7.1; 435/7.92

(57) ABSTRACT

The invention relates to a method for diagnosticating hepatic diseases consisting in measuring an apolipoprotein A4 expression in hepatic cells or tissues and in the circulation (blood, plasma, serum. A method for screening molecules for treating said diseases by bringing into contact said compounds with a mammal and for measuring the apolipoprotein A4 expression in said hepatic origin cells or in the circulation of said mammal is also disclosed.

The present application relates to a method of diagnosing liver diseases. It also relates to a method of screening molecules for the treatment of said diseases.

Apolipoprotein A4 (Apo A4) is an abundant circulating apolipoprotein. The expression of this protein has been demonstrated in rodents in the hypothalamus, the small intestine and the liver. Most of the Apo A4 found in the plasma in mice is considered to be of intestinal origin (Wu et al., JBC 254, 7316-7322 1979).

In humans, the synthesis of Apo A4 has been described by Elshourbagy (JBC 262 1987) as being limited to the small intestine.

Apo A4 has many functions. It regulates lipid transport and metabolism by means of various mechanisms, such as the activation of cholesterol fluxes from hepatic tissues and the stimulation of lipoprotein lipase activity (Stan et al., Biochim Biophys acta, 1631 2003, 177-187). Apo A4 is also known to be a satiety factor.

Liver diseases constitute a major public health problem. It is therefore essential to diagnose all liver diseases at as early a stage as possible, using specific and sensitive assaying methods.

The applicant has demonstrated, surprisingly, that Apo A4 is expressed in the liver in humans and that the level of hepatic expression of the gene encoding Apo A4 is significantly increased in liver diseases.

A subject of the present invention is a method of detecting, diagnosing or providing a prognosis for a liver disease, comprising the measurement of the expression of Apo A4 in hepatic cells or tissues or extracts of these cells and tissues, or in the blood and derivatives thereof.

Thus, a subject of the present invention is the measurement of the expression of Apo A4, not only in hepatic cells or tissues, but also in the blood, in which Apo A4 of hepatic origin is found.

Such a method can comprise the steps of:
isolating the hepatic cells or tissues or extracts of these cells and tissues, or the blood or derivatives thereof, and
measuring the expression of Apo A4.

Such extracts can be obtained by lysis of the hepatic cells and tissues. The term “liver disease” is intended to mean any disease that has an influence on hepatic tissues and cells, whether it is chronic or aetiological (alcoholic, viral, toxic, food-related, environmental, etc).

More particularly, a subject of the present invention is a method of detecting, diagnosing or providing a prognosis for steatohepatitis, liver carcinogenesis, cirrhosis, viral hepatitis, hepatocellular insufficiency, cholestasis, portal hypertension, steatosis and steatohepatitis, hepatic vein and artery disease, fibrosis, obesity, and metabolic syndromes.

The term “blood derivative” is intended to mean any cellular or acellular fraction derived from the blood by physical treatment (for example, by centrifugation), biological treatment (for example, by clotting) or chemical treatment or by any other treatment for obtaining such derivatives.

Such derivatives are preferably plasma and serum.

According to a preferential embodiment of the invention, said method comprises the measurement of the amount of messenger RNAs (mRNAs) transcribed from the gene(s) encoding Apo A4 in the hepatic cells or tissues of an individual in whom it is desired to diagnose or provide a prognosis for a liver disease.

The hepatic cells or tissues or the cells in the blood and derivatives thereof, of said mammal, are removed and treated by methods known to those skilled in the art, in order, in particular, to prevent degradation of the messenger RNAs and of the proteins, and then the amount of messenger RNAs transcribed by the gene(s) encoding Apo A4 is measured.

Particularly advantageously the amount of messenger RNAs transcribed is measured by amplification according to the method known as quantitative polymerase chain reaction (PCR) or QPCR.

According to this technique, the total RNAs are extracted and purified and the messenger RNAs contained in the extract are converted, firstly, into complementary DNAs (cDNAs) using a reverse transcriptase.

The polymerase preferably used in the present invention is Taq polymerase, but it may be any other enzyme that has polymerase activity and that can be used under the conditions for carrying out the PCR. By virtue of its properties, this enzyme makes it possible to double the amount of initial DNA at each synthesis cycle.

The analysis of the cDNAs derived from the mRNAs contained in the biological sample is carried out by real-time quantitative PCR. The cDNAs are amplified using primers and probes specific for the Apo A4 sequence, according to a method which comprises, in substance, a repetition of the cycle comprising the following steps:
separation of the strands to be amplified by heating the cDNA prepared from the sample,
hybridization of the probe and of a pair of sense and antisense primers, and
de novo synthesis of DNA strands by elongation with a polymerase.

Advantageously, the sense and antisense primers comprise at least 15 nucleotides and exhibit at least 80%, preferably 90%, and more preferably 95% identity with the human Apo A4 sequence, corresponding to the sequence SEQ ID No. 13 (Gene bank No. NM 000482), or with the sequence complementary thereto.

In the course of the amplification reaction, the reaction product, or amplimer, is detected using a probe consisting of an oligonucleotide comprising at least 15 nucleotides and exhibiting at least 80%, preferably 90%, and more preferably 95% identity with the human Apo A4 sequence, corresponding to the sequence SEQ ID No. 13 (Gene bank No. NM 000482), or with the sequence complementary thereto.

The two primers are respectively chosen on the sense and antisense strands, so as to allow the amplification of a DNA fragment. The probe is, for its part, targeted so as to hybridize with the DNA fragment resulting from the amplification delimited by the position of the two primers.

Advantageously, the probe has a theoretical melting temperature Tm that is approximately 10° C.±0.5 higher than the theoretical Tm values of the primers. Said oligonucleotides (primers and probe) preferably comprise between 15 and 25 nucleotides. The method is carried out for a number of cycles sufficient to allow a measurable amount of amplification product to be obtained (n=40).

The primers used for amplifying the gene encoding human Apo A4 preferably have the sequences:

SEQ ID No. 1: gcagctggctccctatgct
and
SEQ ID No. 2: ggaaggtcaggccctcaag.

The probe preferably has the sequence SEQ ID No. 3: cacgcaggagaagctcaaccacca.

According to a preferential embodiment of the present invention, the probe contains a visualizing molecule or system of molecules. Said visualizing system preferably consists of a reporter colorant and a fluorescence-quenching colorant, respectively attached at the 5′ and 3′ ends of the probe. According to an advantageous embodiment, the visualizing system consists of the “reporter/quencher” pair represented by 6-carboxyfluorescein (FAM) and 6-carboxytetramethylrhodamine (TAMRA), respectively attached in the 5′-position and in the 3′-position of the probe.

In order to carry out the PCR in the context of the present invention, reference may be made to the general reviews of PCR techniques and to the instructions from the manufacturers and distributors of reagents and thermocycles, and in particular to the instructions for use entitled “Quantitation of DNA/RNA using real-time PCR detection” published by Perkin Elmer Applied Biosystems (1999) and to the PCR Protocols (Academic Press New York 1989).

One of the advantages of the method of detection by QPCR is that the analysis of the PCR products is carried out directly at the end of the PCR cycles by reading the fluorescence obtained during the cycles. It is therefore not necessary to work with the PCR products, which risk being contaminated for the subsequent analyses.

In addition, the quantification of the number of targets used at the beginning in the reaction is very reliable and reproducible. The PCR product is detected during the PCR cycles by means of a fluorescent probe. The latter is necessary for detecting the PCR product and the detection takes place right in the middle of the exponential PCR phase and not at the final point; this principle of detection is therefore more sensitive and more specific.

Another advantage of QPCR lies in the fact that non-specific amplifications are avoided, due to the “hot start” principle, the real-time PCR being carried out in the presence of a thermostable DNA polymerase that is activated at the first denaturation.

According to another embodiment of the invention, the method according to the present invention comprises the measurement of the amount of Apo A4 protein or protein fragments expressed in the hepatic cells or tissues or in the blood and derivatives thereof.

Particularly advantageously, the amount of Apo A4 protein or protein fragments is measured using at least one antibody specific for this protein.

These antibodies can be obtained by means of a method consisting in injecting animals, such as rodents, with an emulsion of the purified human Apo A4 protein or a peptide sequence of Apo A4 of approximately 20 amino acids, optionally in the presence of an adjuvant such as Freund's adjuvant. The injections are repeated and the antiserum is collected.

The measurement of the binding between these antibodies and the Apo A4 expressed in the hepatic cells or tissues or in the blood and derivatives thereof of the individuals in whom it is desired to quantify the hepatic Apo A4 can be carried out by any suitable means. Preferably, it can be carried out by means of the ELISA (abbreviation of enzyme-linked immunosorbent assay) technique, and even more preferably by means of a “sandwich” method. The “sandwich” method makes use of two antibodies: a first capture antibody is bound to a solid phase such as a microplate, and a second detection antibody makes it possible to quantify the protein. According to this technique, the detection antibodies are coupled to a peroxidase. The antibodies bound to the Apo A4 protein are isolated from the unbound antibodies and brought into contact with a substrate for peroxidase, preferably O-phenylenediamine dihydrochloride. The colorimetric reaction makes it possible to quantify the number of antibodies bound and, as a result, the amount of protein bound. This colorimetric reaction is measured using a suitable device, for example by measuring the optical density.

An amplification of the reaction can be carried out using at least two antibodies, firstly anti-Apo A4 antibodies not coupled to peroxidase and, secondly, peroxidase-coupled antibodies that recognize the first antibodies.

In addition, another anti-Apo A4 antibody can be used to attach the antibody-Apo A4 complexes to a support, thus facilitating isolation of the complexes and separation from the unbound antibodies.

The binding between these antibodies and the Apo A4 can also be measured using antibodies labelled with a radioactive isotope. In this embodiment, the antibodies bound to the Apo A4 protein are isolated and the radioactivity of the antibody-Apo A4 complexes is measured using a measuring device suitable for the isotope used.

Techniques other than the ELISA and radioisotope technique can be used for assaying the Apo A4 protein, such as nephelometry and turbidimetry.

For the production of the antibodies and the use of the ELISA and radioisotope techniques in particular, reference may be made to the manual “Antibodies, A Laboratory Manual,” (Cold Spring Harbor Press (1988)).

A subject of the present invention is also a method of screening compounds for use in the prevention or treatment of liver diseases, comprising the steps of:
bringing said compounds into contact with a mammal, and
measuring the expression of Apo A4 in said cells of hepatic origin or in the bloods or in the blood and the blood cell derivatives of said mammal.

The mammal in whose cells or blood the measurement of the expression of Apo A4 can be carried out is any mammal in which Apo A4 is expressed in the liver. It may advantageously be a rodent, and preferably a mouse.

Advantageously, animal lines in which there is a natural tendency towards obesity are used.

Thus, a mouse of a Balbc ByJ, DIO Balbc ByJ, DIO Balbc AnN or DIO C57Bl/6J line is preferably used.

The measurement of the expression of Apo A4 can be carried out by measuring the amount of Apo A4 expressed in the hepatic cells or tissues or in the blood and derivatives thereof of said mammal or by measuring the amount of messenger RNA transcribed by the gene(s) encoding Apo A4 expressed in the hepatic cells or tissues of said mammal, as indicated above.

The present invention is illustrated, without however being limited, by the examples of implementation that follow.

### EXAMPLE 1

### Measurement of the Expression of Apo A4 in Normal Mice (C57 Bl6) or Obese Mice (C57 Bl6/obob) by Quantification of the Messenger RNAs Transcribed and of the Plasma Apo A4 Protein

1. Materials and Methods

### Experimental Protocol

Blood was taken from the normal mice (C57 Bl6) or obese mice (C57 Bl6/obob) and centrifuged for 10 min at 10 000 rpm and the plasma was removed.

A piece of liver (75-130 mg) was removed and placed in tubes of 2 ml containing 1.5 ml of RNA Later (Ambiom).

The liver and the plasma were stored at −20° C. with a view to assaying the Apo A4 mRNAs and protein.

### Real-Time QPCR Analysis of the Expression of Mouse Apo A4

This mainly 2-step technique involves:

1. Extraction and purification of the total RNAs and then conversion of the mRNAs contained in the sample into complementary DNA (cDNA).
2. Analysis of the cDNAs, carried out by real-time quantitative PCR. The relative expressions of the gene are given as % relative to an internal reference gene that does not vary, such as beta2-microglobulin or 18S ribosomal RNA.

The 7900HT Fast Real-Time PCR System sequence analyser and the reagents for the amplification are provided by Applied Biosystems.

### Methods:

### Preparation of Tissue Lysates

80 mg of mouse liver are excised and immediately immersed in 2 ml of RNAlater in order to preserve as much as possible the integrity of the RNA before any manipulation.

The piece of liver is transferred dry into an Eppendorf tube, to which are added 2 ml of lysis buffer (total RNA extraction kit, RNeasy Midi Kit sold by QIAGEN) and 2 steel balls (diameter, 3 mm). The tissue lysis is carried out with the Tissuelyser (sold by QIAGEN) by agitation for 5 min at 30 HZ. At this stage, the lysates can be stored at −20° C.

### Total RNA Extraction

The lysates are centrifuged for 3 min at 14 000×g and filtered on a QIAshredder (sold by QIAGEN) (3×0.7 ml). The extraction is carried out according to the protocol of the RNeasy Midi kit, with a Dnase step. The final step is an elution of the total RNA in 150 μl of water.

The RNA concentration is obtained by measuring the optical density (OD) at 260 nm.

Complementary DNA (cDNA) Synthesis

The cDNA is synthesized, from 5 μg of total RNA, using the Superscript III reverse transcription kit (sold by INVITROGEN). The cDNA obtained is recovered in a final volume of 20 μl.

### Analysis of the Gene Expression by PCR on 7900HT

5 μl of a dilution of cDNA are added to 15 μl of reaction mixture containing, in particular: buffer, polymerase required for the PCR reaction and also the primers and probes specific for the gene to be quantified. The latter are available from the supplier in the form of Taqman Gene Expression Assays. The beta2-microglobulin (B2-m) gene or 18S ribosomal RNA are used as internal references.

The sequences of the primers and probes used for amplifying the gene encoding human and mouse Apo A4 and beta2-m are represented in Table VII (SEQ ID No. 1 to SEQ ID No. 12).

The gene amplification reaction is carried out with temperature cycles (denaturation 95° C./15 s then hybridization and synthesis 60° C./1 min) in 96-well or 384-well microplates. The analysis lasts 90 min.

The amplification kinetics (amplification signal as a function of the number of cycles) are analysed in a linear representation of the exponential phase (software: SDS Enterprise Database).

The Ct, which is the number of cycles measured at constant amplification signal, is defined in this phase; the Ct is inversely proportional to the amount of messenger RNA present in the sample of origin. The difference in Ct (ΔCt) between the target gene (Apo A4) and the internal reference (B2-m) gives the relative abundance from the relationship Q=(2)^{-ΔCt} (values given as % relative to the reference gene).

### Measurement of the Amount of Apo A4 in the Plasma in Mice, Using the ELISA Sandwich Method

### Antibodies

The capture antibody is the goat anti-Apo A4 antibody sold by (Santa Cruz Biotechnology, Inc. 2145 Delaware Avenue Santa Cruz, Calif. 95060 U.S.A.).

Antiserum directed against mouse Apo A4 is obtained from rabbits immunized with a peptide corresponding to the C-terminal sequence (amino acids 361-380) of mouse Apo A4.

An emulsion of peptide coupled to a carrier protein (KLH) in complete Freund's adjuvant is injected subcutaneously into the animals. Subsequent injections are given with an emulsion of coupled peptide in incomplete Freund's adjuvant.

The antiserum is collected 10 days after the third booster and the antibody is purified by affinity chromatography on the peptide coupled to a chromatography gel.

### Implementation of the ELISA Sandwich Method

100 μl of goat anti-Apo A4 antibody at 4 μg/ml in PBS are incubated for 4 hours at 20° C., in the wells of a 96-well plate.

After washing with PBS containing 0.1% of tween 20, the non-specific binding sites are blocked with PBS buffer containing 0.5% of bovine serum albumin (BSA) for 60 min at ambient temperature.

100 μl of plasma samples (dilution in PBS containing 0.1% BSA, of 1/3000 to 1/20 000) are added and incubated overnight at 4° C. After washing, 100 μl of rabbit polyclonal anti-mouse Apo A4 antibodies, at 0.1 μg/ml, are added and incubated for 1 h at ambient temperature. After washing, 100 μl of anti-rabbit IgG-peroxidase conjugates (PIERCE 1/200 in PBS containing 0.1% BSA) are added and incubated for 1 h at ambient temperature.

After washing, 150 μl of a solution containing O-phenylenediamine and hydrogen peroxide, which is the substrate for peroxidase (Sigma), are added.

After 20 min, 50 μl of 3M H₂SO₄ are added in order to stop the reaction, and the optical density at 492 nm is measured.

2. Results:

Table IA shows the tissue distribution of Apo A4 in the mouse. Apo A4 is expressed in smooth muscle, small intestine, liver, colon, placenta, stomach, ovaries, rectum, adipose tissues, etc.

Strong expression in the small intestine is noted.

Table IB confirms strong expression in the small intestine and the location of Apo A4 in the various regions (duodenum, jejunum, ileum).

Table II shows the expression of Apo A4 in the liver and the plasma and shows that this expression is increased in the liver and the plasma if normal mice and obese mice are compared.

### EXAMPLE 2

### Measurement of the Expression of Apo A4 in Humans, by Quantification of Transcribed Messenger RNAs

The cDNAs are prepared from human liver and measurements are carried out as described in the previous example for the preparation from mouse liver.

### Results of the Real-Time QPCR Analysis of Human Apo A4 Expression

Table III shows the tissue distribution of Apo A4 in humans: presence in the uterine fundus, oesophagus, retina, placenta, epididymis and adipose tissues.

Strong expression is noted in the small intestine, in agreement with the data from the literature.

There is little expression in the total liver, but said expression appears to be concentrated on the hepatoportal region.

Table IV-A confirms the results on the location of Apo A4 in the regions of the small intestine (jejunum, ileum, duodenum).

It also emerges that the local expression of Apo A4 in the liver is more associated with certain physiopathological states (hepatic portal system) (Table IV-B).

This result is confirmed in Tables V-A & VI, which group together an analysis of Apo A4 on various samples of pathological livers and of normal liver.

The high values are correlated for the following pathologies: cirrhosis, lupus and infarction.

The specificity of Apo A4 as a potential marker for these conditions is demonstrated by the analysis, compared with Apo A4 of the other classes of apolipoproteins: Apo A1, Apo A2 and Apo A5 (Tables IV and V-B).

### EXAMPLE 3

### Measurement of the Amount of Apo A4 Protein in the Plasma in Humans, by Means of the ELISA Sandwich Method

### Antibodies

The capture antibody is the goat anti-Apo A4 antibody sold by (Santa Cruz Biotechnology, Inc. 2145 Delaware Avenue Santa Cruz, Calif. 95060 U.S.A.).

Purified human Apo A4 is obtained from human serum as described by Weinberg R B, Hopkins R A, Jones J B. (Methods Enzymol. 1996; 263:282-96. Purification, isoform characterization, and quantitation of human apolipoprotein A4).

Antiserum directed against human Apo A4 is obtained from rabbits. An emulsion of purified h-Apo A4 in complete Freund's adjuvant is injected subcutaneously into the animals. Subsequent injections are given with an emulsion of purified Apo A4 in incomplete Freund's adjuvant.

The antiserum was collected 10 days after the third booster.

### Implementation of the ELISA Sandwich Method

The wells of a 96-well plate are incubated with 100 μl of goat anti-Apo A4 antibody at 4 μg/ml in PBS for 4 hours at 20° C.

After washing with PBS containing 0.1% of tween 20, the non-specific binding sites are blocked with PBS buffer containing 0.5% of bovine serum albumin (BSA) for 60 min at ambient temperature.

100 μl of plasma samples (dilution in PBS containing 0.1% BSA, of 1/3000 to 1/20 000) are added and incubated overnight at 4° C. After washing, 100 μl of rabbit anti-human Apo A4 polyclonal serum antibodies (dilution 1/5000 in PBS-BSA) are added and incubated for 1 h at ambient temperature. After washing, 100 μl of anti-rabbit IgG-peroxidase conjugates (PIERCE, 1/200 in PBS containing 0.1% BSA) are added and incubated for 1 h at ambient temperature. After washing, 150 μl of a solution containing O-phenylenediamine and hydrogen peroxide, which is the substrate for peroxidase (Sigma), are added.

After 20 min, 50 μl of 3M H₂SO₄ are added in order to stop the reaction, and the optical density at 492 nm is measured.

### EXAMPLE 4

### Obesity and Human Hepatic apoA4

A study focused on obesity was carried out by analysing the expression of apoA4 in the liver of overweight patients. This pathology is correlated with the body mass index (BMI=weight in kg/(height in metres)²). A normal weight load corresponds to a BMI<25.

Experimentally, the apolipoprotein expression was analysed by means of the QPCR technique described above, using a collection of human liver RNAs provided by Asterand, Inc. (TechOne Bldg, Suite 501, 440 Burroughs, Detroit Mich. 48202, US). The body mass indices (BMIs) are given by Asterand.

The results are given in Table VIII.

A direct positive relationship is noted between the % expression of apoA4 and the body mass index.

These results demonstrate the relevance of the use of apoA4 as a marker in the diagnosis or prognosis and therapeutic follow-up of obesity.

TABLE IA
Expression of Apo A4 in mice (expressed
as % relative to beta2-microglobulin)

| Tissues/organ | apoA4 % |
|---|---|
| Embryo | 198 |
| Smooth muscle | 189 |
| Small intestine | 174 |
| Liver | 172 |
| Colon | 79 |
| Placenta | 75 |
| Stomach | 46 |
| Ovary | 7.1 |
| Rectum | 2.1 |
| Adipose tissue | 0.7 |
| Brain | 0.6 |
| Testes | 0.3 |
| Thymus | 0.3 |
| Kidney | 0.2 |
| Spleen | 0.1 |
| Bladder | 0.1 |
| Thyroid | 0.1 |
| Skeletal muscle | 0.1 |
| Spinal cord | 0.04 |
| Lymph node | 0.03 |
| Prostate | 0.02 |
| Eye | 0.01 |
| Lung | 0.004 |
| Splenocytes | 0.0001 |
| Bone marrow | 0 |
| Heart | 0 |

TABLE IB
Expression of Apo A4 in the regions of the small intestine
(%/beta2-m, mean +/− standard deviation (4 animals))

| Region | Apo A4 % |
|---|---|
| Duodenum | 213 +/− 19 |
| Jejunum | 209 +/− 101 |
| Ileum | 3 +/− 1 |

TABLE II
Expression of Apo A4 mRNA in the liver and measurement of Apo A4
in the plasma of normal mice (C57 BI6) or obese mice (C57 BI6/obob).
Liver Apo A4 mRNA % expression/b2m

| | | SD |
|---|---|---|
| C57 BI6 mice | 2 | 1 |
| C57 BI6/obob obese | 224 | 68 |

Plasma Apo A4 (arbitrary units)

| | |
|---|---|
| C57 BI6 mice | 1 |
| C57 BI6/obob obese | 5 |

TABLE III
Human Apo A4 expression in tissues

| Tissues | Apo A4 % |
|---|---|
| Uterine fundus | 1 |
| Uterine corpus | 0.03 |
| Uterus | 0 |
| Tonsils | 0.004 |
| Oesophagus | 1.2 |
| Gall bladder | 0.01 |
| Maxillary gland | 0 |
| Vein | 0.03 |
| Artery | 0.2 |
| Breast | 0.006 |
| Penis | 0 |
| Skin | 0.03 |
| Brain striatum | 0 |
| Larynx | 0.01 |
| Retina | 4 |
| Mammary gland | |
| Smooth muscle | 0 |
| Spinal cord | 0.8 |
| Aorta | 0.02 |
| Lung (polyA) | 0.2 |
| Placenta (polyA) | 1 |
| Brain | 0.1 |
| Epididymis (polyA) | 1 |
| Adipose tissues (polyA) Clontech | 3 |
| Hepatoportal triad liver | 4980 |
| Liver left lobe | 1 |
| Liver median lobe | 0 |
| Liver right lobe | 0 |
| Liver | 0.4 |
| Testes | 0.2 |
| Rectum | 0.1 |
| Carotid artery | 0 |
| Adrenal gland | 0.01 |
| Prostate | 0.03 |
| Adipose tissues biochain | 0 |
| Spleen | 0 |
| Colon | 0.02 |
| Pancreas | 0.01 |
| Heart | 0.01 |
| Umbilical vein | 0 |
| Stomach | 0.05 |
| Thymus | 0.1 |
| Uterine cervix | 0 |
| Skeletal muscle | 0 |
| Trachea | 0.002 |
| Kidney | 0.005 |
| Lymph node | 0.2 |
| Ovarian tumour | 0 |
| Pericardium | 0.01 |
| Ovary | 0 |
| Bladder | 0 |
| Thyroid | 0.2 |
| Bone marrow | 0 |
| Small intestine duodenum | 0.3 |
| Small intestine | 100 |

TABLE IVA
Apolipoprotein expression in the intestine

| | Apo A1 | Apo A2 | Apo A4 | Apo A5 |
|---|---|---|---|---|
| Tissues/organ | % | % | %/intestine | ° % |
| Small Intestine | 0.1 | 0.0001 | 100 | 0.0001 |
| (new) | | | | |
| Small intestine | 3.4 | 0.0004 | 1347 | 0.003 |
| ileum | | | | |
| Small intestine | 3.5 | 0.001 | 1559 | 0.002 |
| jejunum | | | | |
| Small intestine | 0.0002 | 0 | 0.05 | 0 |
| duodenum | | | | |

TABLE IVB
Results grouped together for the liver

| | | | Apo A4 | Apo A5 | Medical | Reasons for | |
|---|---|---|---|---|---|---|---|
| | Apo A1 % | Apo A2 % | %/intestine | ° % | history | death | Age |
| Hepatic | 2.3 | 3 | 0.2 | 0.5 | Hypertension | Acute | 33 |
| portal | | | | | | myocardial | |
| system 12 | | | | | | infarction | |
| Hepatic | 2 | 3.5 | 531 | 2 | Emphysema | Acute | 69 |
| portal | | | | | | myocardial | |
| system 16 | | | | | | infarction | |
| Left lobe | 1.5 | 2 | 0.14 | 0.3 | Hypertension | Cardiomegaly | 71 |
| liver 13 | | | | | | | |
| Left lobe | 0.9 | 1.3 | 0.2 | 0.2 | No history | Acute | 73 |
| liver 17 | | | | | | myocardial | |
| | | | | | | infarction | |
| Median | 14 | 15 | 54 | 0.3 | Attack, | Acute | 48 |
| lobe liver | | | | | hypertension | myocardial | |
| 14 | | | | | | infarction | |
| Median | 0.0005 | 0 | 0 | 0.0006 | Hypertension | Left | 61 |
| lobe liver | | | | | | ventricular | |
| 18 | | | | | | hypertrophy | |
| Right lobe | 0.0004 | 0 | 0 | 0.0001 | Hypertension | Left | 55 |
| liver 15 | | | | | | ventricular | |
| | | | | | | hypertrophy | |
| Right lobe | 0.02 | 0.004 | 0 | 0.002 | Hypertension | Adult | 21 |
| liver 19 | | | | | | respiratory | |
| | | | | | | distress | |
| | | | | | | syndrome | |
| Total liver | 2.5 | 3 | 0.01 | 0.5 | | | |

TABLE VA
Human Apo A4 expression in the liver samples

| | | Histological | |
|---|---|---|---|
| Sample | Origin | diagnosis | Apo A4 % |
| Liver | Biochain | Normal | 0.07 |
| Right lobe liver | Biochain | Normal | 0.2 |
| Left lobe liver | Biochain | Normal | 0.8 |
| Liver cirrhosis | Biochain | Cirrhosis | 7.6 |
| Lupus liver disease | Biochain | Lupus | 15.3 |
| Foetal liver | Biochain | Normal | 0.8 |
| Liver tumour | Biochain | Hepatocellular | 0.02 |
| | | carcinoma | |
| Small intestine | Biochain | Normal | 100 |
| Hepatic portal system | Clinomics 05-22 | Cirrhosis | 9.4 |
| Left lobe liver | Clinomics 05-23 | Cirrhosis | 8.6 |
| Median lobe liver | Clinomics 05-24 | Cirrhosis | 12.7 |
| Right lobe liver | Clinomics 05-25 | Cirrhosis | 8.3 |

TABLE VB
Expression of human Apo A1, 2 & 5 in the liver samples

| Sample | Apo A1/18S % | Apo A2/18s % | Apo A5/18S % |
|---|---|---|---|
| Liver | 27 | 32 | 0.5 |
| Right lobe liver | 15 | 22 | 0.6 |
| Left lobe liver | 20 | 34 | 0.8 |
| Liver cirrhosis | 2 | 3 | 0.08 |
| Lupus liver disease | 9 | 11 | 1 |
| Foetal liver | 20 | 30 | 0.04 |
| Liver tumour | 0.5 | 22 | 0.1 |
| Small intestine | 0.5 | 0 | 0 |
| Hepatic portal system | 1 | 2 | 0.04 |
| Left lobe liver | 1 | 3 | 0.06 |
| Median lobe liver | 0.8 | 1.5 | 0.05 |
| Right lobe liver | 0.7 | 1.5 | 0.04 |

TABLE VI
Expression of Apo A4 in the human samples

| Sample | Status | Reasons for death | Apo A4 % |
|---|---|---|---|
| Hepatic portal system 12 | Hypertension | Acute myocardial | 0.2 |
| | | infarction | |
| Left lobe liver 13 | Hypertension | Cardiomegaly | 0.14 |
| Median lobe liver 14 | Attack, | Acute myocardial | 54 |
| | hypertension | infarction | |
| Right lobe liver 15 | Hypertension | Left ventricular | 0 |
| | | hypertrophy | |
| Hepatic portal system 16 | Emphysema | Acute myocardial | 531 |
| | | infarction | |
| Left lobe liver 17 | Unknown | Acute myocardial | 0.2 |
| | | infarction | |
| Median lobe liver 18 | Hypertension | Left ventricular | 0 |
| | | hypertrophy | |
| Right lobe liver 19 | Hypertension | Adult respiratory | 0 |
| | | distress syndrome | |
| Total liver | Normal | | 0.01-0.07 |
| Right lobe liver | Normal | | 0.2 |
| Left lobe liver | Normal | | 0.2 |
| Liver cirrhosis | Cirrhosis | | 7.6 |
| Lupus liver disease | Lupus | | 15 |
| Foetal liver | Normal | | 0.8 |
| Liver tumour | Tumour | | 0.02 |
| Hepatic portal system 22 | Cirrhosis | | 9 |
| Lett lobe liver 23 | Cirrhosis | | 9 |
| Median lobe liver 24 | Cirrhosis | | 13 |
| Right lobe liver 25 | Cirrhosis | | 8 |
| Total brain | | | |
| Small intestine | | | 100 |

TABLE VII
Sequences of the primers and of the probes for assaying, by
quantitative PCR, human and mouse beta2-microglobulin and Apo A4

| | | | |
|---|---|---|---|
| 5′-3′ Sense | 5′-3′ Antisense | 5′ Fam-3′ Tamra | |
| primer | primer | probe | Gene bank |

| | | | | |
|---|---|---|---|---|
| Human Apo A4 | SEQ ID No. 1 | SEQ ID No. 2 | SEQ ID No. 3 | NM_000482 |
| | gcagctggctccct | ggaaggtcaggccctc | cacgcaggagaag | |
| | atgct | aag | ctcaaccacca | |
| Mouse Apo A4 | SEQ ID No. 4 | SEQ ID No. 5 | SEQ ID No. 6 | NM_007468 |
| | gggtgcacaaca | ccctctcagtttccttg | ccctttgtcgtacagct | |
| | agctggt | gctag | gagtgggca | |
| Human beta2-m | SEQ ID No. 7 | SEQ ID No. 8 | SEQ ID No. 9 | NM_004048 |
| | gatgagtatgcctg | ggcatcttcaacctcca | aaccatgtgactttgt | |
| | ccgtgt | tga | cacag | |
| Mouse beta2-m | SEQ ID No. 10 | SEQ ID No. 11 | SEQ ID No.12 | NM_009735 |
| | acgccacccacc | ggcgggtggaactgtg | tgggaagccgaaca | |
| | gga | tta | tactgaactgctacg | |

TABLE VIII
Expression of human hepatic apoA4 in overweight patients

| Sample | BMI | apoA4 % |
|---|---|---|
| 8091D1 | 41.87 | 115 |
| 9765C1 | 39.3 | 19 |
| 4107B1 | 35.32 | 1 |
| 17061F1 | 33.43 | 41 |
| 11631B1 | 33.08 | 4.1 |
| 9182B1 | 32.05 | 31 |
| 5412B1 | 31.23 | 6 |
| 20211A1 | 30.06 | 2 |
| 15140F1 | 29.86 | 3.3 |
| 6213B1 | 29.82 | 4 |
| 14094A1 | 27.77 | 1.6 |
| 8554D1 | 26.81 | 3.2 |
| 20235A1 | 26.23 | 10 |
| 14050A1 | 26.12 | 0.6 |
| 9574B1 | 25.39 | 3 |
| 19844A1 | 25.25 | 0.2 |
| 5417B1 | 25.21 | 0.15 |
| Normal liver | | 0.04 |

1. A method of diagnosing or providing a prognosis for liver disease, comprising the measurement of the expression of Apo A4 in hepatic cells or tissues or in the blood and derivatives thereof.

2. A method of diagnosing or providing a prognosis for liver disease, comprising
isolating the hepatic cells or tissues or extracts of these cells and tissues, or the blood or derivatives thereof, and
measuring the expression of Apo A4.

3. A method according to claim 2 characterized in that it comprises the measurement of the amount of messenger RNAs transcribed by the gene(s) encoding Apo A4, expressed in the hepatic cells or tissues.

4. The method according to claim 3, characterized in that the amount of messenger RNAs transcribed is measured by means of the PCR method.

5. The method according to claim 4, characterized in that the amount of messenger RNAs transcribed is measured by means of the quantitative PCR method.

6. The method according to claim 2 characterized in that it comprises the measurement of the amount of Apo A4 protein expressed in the hepatic cells or tissues or in the blood and derivatives thereof.

7. The method according to claim 6, characterized in that the amount of Apo A4 is measured using at least one antibody specific for this protein.

8. The method according to claim 7, characterized in that the specific antibody is coupled to a peroxidase or is labelled with a radioactive isotope.

9. The method according to either of claims 7 and 8, characterized in that the binding of the antibody to the protein is measured by the ELISA technique.

10. A method of screening or detecting compounds for use in the prevention or treatment of liver diseases, comprising the steps of:
bringing said compounds into contact with a mammal, and
measuring the expression of Apo A4 in the cells of hepatic origin or in the blood and blood derivatives of said mammal.

11. The method according to claim 10, characterized in that it comprises the measurement of the amount of Apo A4 expressed in the hepatic cells or tissues or in the blood and blood derivatives of said mammal.

12. The method according to claim 10, characterized in that it comprises the measurement of the amount of messenger RNAs transcribed by the gene(s) encoding Apo A4 expressed in the hepatic cells or tissues or in the blood and blood derivatives of said mammal.

13. The method according to claims 10-12 in the alternative characterized in that said mammal is a mouse of the Balbc ByJ, DIO Balbc ByJ, DIO Balbc AnN or DIO C57Bl/6J line.
